(19) United States
(12) Patent Application Publication
(10) Pub. No.: US 2008/0132688 A1
(43) Pub. Date: Jun. 5, 2008

(54) Methods for Removing Viral Contaminants During Protein Purification

(75) Inventor: Joe Zhou, Westlake Village, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMGEN INC., Thousand Oaks, CA (US)

(21) Appl. No.: 11/859,234

(22) Filed: Sep. 21, 2007

Related U.S. Application Data
(60) Provisional application No. 60/846,611, filed on Sep. 22, 2006.

(51) Int. Cl. C07K 1/02

(52) U.S. Cl. 530/413; 530/412; 530/427

(57) ABSTRACT

The present invention relates, in general, to methods for removing viral contaminants from therapeutic protein solutions to improve safety of therapeutic proteins administered to patients. Particularly contemplated is the removal of small non-enveloped viruses, such as parvovirus, from therapeutic protein solutions.

### CROSS-REFERENCE TO RELATED APPLICATIONS

The present application claims the priority benefit of U.S. Provisional Application No. 60/846,611, filed Sep. 22, 2006, herein incorporated by reference in its entirety.

### FIELD OF THE INVENTION

The present invention relates, in general to methods for removing viral contaminants during manufacturing of therapeutic proteins.

### BACKGROUND OF THE INVENTION

The use of recombinantly produced therapeutic proteins has continued to increase in importance as methods of treating many diseases or conditions that affect individuals, such as cancer and autoimmune diseases (Daemmrich et al., Chem Eng News, June, 28-42 (2005); Chadd, et al., Curr Opin Biotech 12:188-94 (2001); Walsh, G. BioPharm International 18, 58-65 (2005)). However, large-scale production of these protein therapeutics still remains a challenge (Li, et al., Bioprocessing J. 4:23-30 (2005)). For example, the commercial manufacturing process must deliver a reliably high-yield with downstream processes producing an extremely pure product allowing only trace amounts, to preferably, no contaminants.

Chinese hamster ovary (CHO) mammalian cell lines serve as efficient expression systems for the production of protein therapeutics (Chu et al., Curr. Opin. Biotech 12:180-87 (2001)). However, mammalian cell systems are susceptible to contamination with adventitious viruses that may be introduced through raw materials or failures in process controls. Partial physico-chemical and biological characteristics of different viruses that can infect mammalian cells are listed in Table 1. All viruses contain nucleic acid, either DNA or RNA, surrounded by a protective protein coat called a capsid. Some viruses are also enclosed by an envelope of lipid and protein molecules that is derived from the host cell membrane but includes virus proteins. Numerous types of viruses can infect mammalian cells, including RNA and DNA viruses, which may be enveloped or non-enveloped (“naked”). In addition, non-infectious retrovirus-like particles are produced by CHO cells and are consistently observed and quantitated by electron microscopy (Anderson et al., J. Virol. 64:2021-2032 (1990); Anderson et al., Virology 181:305-11 (1991)). Because of this, model and relevant viruses that are readily detected and quantitated in these cell cultures are used to characterize potential protein purification processes for their capacity to clear adventitious viral agents.

Xenotropic murine leukemia virus (x-MuLV) is a large (80-130 nm) enveloped, RNA virus belonging to the Retroviridae family of viruses. In viral clearance studies, x-MuLV is used as model virus in determining the capacity of the purification process for clearance of the non-infectious retroviral-like particles produced by CHO cells.

Murine minute virus (MMV) (or minute virus of mice, MVM) is a non-enveloped single-strand DNA virus with an average size of 18-26 nm. MMV is a member of the Parvoviridae family, which have been shown to be resistant to heat, detergents, organic solvents, and exposure to pH 3-11.8 (Boscheti et al., Biologicals 31:181-85 (2003)). Like other parvoviruses, MMV is highly resistant to physiochemical treatment. For example, MMV has been shown to remain active after exposure to pH 4 for 9 hours (Boschetti et al., Transfusion 44:1079-86 (2004)). MMV can adventitiously infect CHO cells during the process of culturing protein therapeutics or the process of purifying the proteins from culture. This high resistance of MMV to inactivation during the purification processes poses a threat to the production of protein therapeutics (Garuick, R., Dev Biol Stand. 88:49-56 (1996); Garuick, R., Dev Biol Stand. 93:21-29 (1998)). In viral clearance studies, MMV is used as a relevant model for small, highly resistant viruses.

X-MuLV and MMV are common model viruses used to test the viral clearance efficiency of each unit operation during recombinant protein purification (Shi, L. et al. Biotech. Bioeng. 87:884-896 (2004); Bray et al. Monoclonal antibody production: minimizing virus safety issues, Vol. 1. (Plenum Publishers, New York; 2004)).

A common method for removing virus from protein solutions comprises using virus filter membranes which are capable of removing viruses having a greater molecule size than the membrane pore size, e.g. nanofiltration of a nearly purified protein solution. However, when the virus is smaller in size than the pore size viral contaminants leak through. This is a persistent problem with parvovirus, which is also highly resistant to physicochemical inactivation. Additionally, the use of a virus-specific membrane having too small a pore size results in clogging with the sample being filtered, which makes filtration difficult. Furthermore, lower flow rates caused by such clogging in parallel with the large sample amounts to filter give rise to many problems, such as limited sample amount to be treated and a longer treatment time.

Common methods of viral inactivation, for example, treatment with chemicals, heat or low pH, are undesirable for use with therapeutic proteins because they may denature and/or aggregate the protein, reducing its biological activity and possibly increasing immunogenic activity. For example, most proteins except for immunoglobulins are damaged by exposure to the acidic conditions needed to kill viruses.

Thus, there remains a need in the art to develop methods for purifying recombinant protein therapeutics minimizing the amount of contamination by viruses during the purification processes.

### SUMMARY OF THE INVENTION

The present invention is directed to a method for removing viral contaminants from purified protein therapeutic solutions.

In one aspect, the invention provides a method for removing virus or fragments thereof from a therapeutic protein solution comprising the step of passing the solution through a depth filter at a pH that is within about 1 pH unit, or within about 0.9, 0.8, 0.7, 0.6, 0.5, 0.4, 0.3, 0.2 or 0.1 pH unit of the isoelectric point of the virus. In one embodiment, the contaminating virus is a parvovirus with a pH of about 5 and the pH is within the range of pH 4 to pH 6. In a further embodiment the pH is about pH 4.8 to 5.2.

The contaminating virus may be a non-enveloped virus. In a related embodiment, the non-enveloped virus is selected from the group consisting of Parvoviridae, Adenoviridae, Birnaviridae, Papovaviridae (e.g., Papillomaviridae and Polyomaviridae), Picornaviridae, Reoviridae and Calciviridae. It is further contemplated that the non-enveloped virus is selected from the group consisting of adenoviruses (e.g. mouse adenovirus-1 and -2), polyoma viruses (e.g. mouse polyoma virus, SV40), hepatitis virus A, polio viruses and parvo viruses (e.g. mouse minute virus, mouse parvovirus), picornaviruses and reoviruses. In one embodiment, the virus is a parvovirus. In a related embodiment, the parvovirus is selected from the group consisting of any mammalian parvovirus, mouse minute virus, mouse parvovirus, porcine parvovirus and human parvovirus.

In exemplary embodiments, the contaminating virus has an average size of less than about 90, 80, 70, 60, 50, 40, or less than about 30 nm.

The depth filtration step according to the invention is preferably not carried out immediately following a viral precipitation step. The depth filtration step can be combined with any other viral inactivation steps or protein purification steps known in the art. Viral inactivation steps include treatment with acid, detergent, solvent, other chemicals, nucleic acid cross-linking agents, UV light, gamma radiation, or heat. Protein purification steps include ion exchange (cation or anion) chromatography, hydrophobic interaction chromatography, size exclusion chromatography, affinity chromatography, dye chromatography, and can be HPLC or reversed phase (e.g. RP-HPLC).

In another aspect, the method of the invention contemplates that specific combinations or sequences of steps are particularly advantageous. Thus, the invention provides that the depth filtration step is combined with a pH inactivation step of maintaining the solution at a pH and for a length of time effective to inactivate virus in the solution. In one embodiment, the pH of the inactivating step is within the range of pH 2.5 to pH 5. In another embodiment, the pH is within the range of pH 2.5-4. In a further embodiment, the pH is within the range of pH 3-4. In a related embodiment, the pH inactivating step is carried out for a length of time from 15 to 90 minutes. In an exemplary embodiment, the pH inactivating step is carried out immediately before the depth filtration step.

The invention further provides that the content of non-enveloped viruses in the therapeutic protein solution is reduced by at least 6, 5, 4, 3, 2 or 1.5 logs after any of the foregoing methods.

In exemplary embodiments, the depth filter comprises diatomaceous materials. In one embodiment, the depth filter is an electropositively charged filter. In one exemplary embodiment, the depth filter is a Millipore A1HC filter or a Cuno ZA series filter.

The methods of the invention may be applied to any therapeutic protein, including erythropoietin, darbepoietin, granulocyte-colony stimulating factor, or an antibody. Antibodies contemplated by the invention include full length antibodies, monoclonal antibodies, polyclonal antibodies, multispecific antibodies (e.g., bispecific antibodies), antibody fragments that can bind antigen (e.g., Fab′, F′(ab)2, Fv, single chain antibodies, diabodies, complementarity determining region (CDR) fragments), and recombinant peptides comprising the forgoing as long as they exhibit the desired biological activity.

The invention also provides that where the therapeutic protein is an antibody, the solution is passed through a protein A affinity chromatography column before being passed through the depth filter. Additional steps for protein purification such as polishing steps are also contemplated. Polishing steps refer to removal of impurities during protein purification using methods, including, but not limited to, cation-exchange chromatography, anion-exchange chromatography, hydrophobic-interaction chromatography, hydroxyapatite chromatography and chromatofocusing.

### BRIEF DESCRIPTION OF THE DRAWINGS

FIG. 1 shows levels of MMV (FIG. 1A) and MuLV (FIG. 1B) in a purified protein solution after low pH inactivation over a period of 70 minutes.

FIG. 2 shows the reduction in MMV levels after depth filtration of a solution containing the virus.

### DETAILED DESCRIPTION

The present invention provides methods for removing viral contaminants during the protein purification process. The methods of the invention are particularly effective for removing small, non-enveloped viruses, such as parvoviruses, that are often difficult to remove and resistant to other methods of virus inactivation. The depth filtration step described herein can provide at least a 3 log (10³) reduction in virus content of the therapeutic protein solution, in a single step. In combination with other steps, the depth filtration step is able to removes such viruses to a significantly greater extent than conventional methods.

The term “therapeutic polypeptide” or “therapeutic protein” refers to any polypeptide or fragment thereof administered to correct a physiological defect including inborn genetic errors, to replace a protein that is not expressed or expressed at low level in a subject or to alleviate, prevent or eliminate a disease state or condition in a subject. The term “therapeutic efficacy” refers to ability to of the therapeutic polypeptide to (a) prevent the development of a disease state or pathological condition, either by reducing the likelihood of or delaying onset of the disease state or pathological condition or (b) reduce or eliminate some or all of the clinical symptoms associated with the disease state or pathological condition. A “therapeutic protein solution” refers to an aqueous solution of therapeutic protein, preferably cell culture media that has been previously subjected to one or more purification steps that separate therapeutic protein from host cell contaminants.

Other examples of proteins include granulocyte-colony stimulating factor (GCSF), stem cell factor, leptin, hormones, cytokines, hematopoietic factors, growth factors, antiobesity factors, trophic factors, anti-inflammatory factors, receptors or soluble receptors, enzymes, variants, derivatives, or analogs of any of these proteins. Other examples include insulin, gastrin, prolactin, adrenocorticotropic hormone (ACTH), thyroid stimulating hormone (TSH), luteinizing hormone (LH), follicle stimulating hormone (FSH), human chorionic gonadotropin (HCG), motilin, interferons (alpha, beta, gamma), interleukins (IL-1 to IL-12), tumor necrosis factor (TNF), tumor necrosis factor-binding protein (TNF-bp), brain derived neurotrophic factor (BDNF), glial derived neurotrophic factor (GDNF), neurotrophic factor 3 (NT3), fibroblast growth factors (FGF), neurotrophic growth factor (NGF), bone growth factors such as osteoprotegerin (OPG), insulin-like growth factors (IGFs), macrophage colony stimulating factor (M-CSF), granulocyte macrophage colony stimulating factor (GM-CSF), megakaryocyte derived growth factor (MGDF), keratinocyte growth factor (KGF), thrombopoietin, platelet-derived growth factor (PGDF), colony simulating growth factors (CSFs), bone morphogenetic protein (BMP), superoxide dismutase (SOD), tissue plasminogen activator (TPA), urokinase, streptokinase, or kallikrein, receptors or soluble receptors, enzymes, variants, derivatives, or analogs of any of these proteins.

Exemplary antibodies are Herceptin® (Trastuzumab), a recombinant DNA-derived humanized monoclonal antibody that selectively binds to the extracellular domain of the human epidermal growth factor receptor 2 (Her2) proto-oncogene; and Rituxan® (Rituximab), a genetically engineered chimeric murine/human monoclonal antibody directed against the CD20 antigen found on the surface of normal and malignant B lymphocytes. Other exemplary antibodies include Avastin® (bevacizumab), Bexxar® (Tositumomab), Campath® (Alemtuzumab), Erbitux® (Cetuximab), Humira® (Adalimumab), Raptiva® (efalizumab), Remicade® (Infliximab), ReoPro® (Abciximab), Simulect® (Basiliximab), Synagis® (Palivizumab), Xolair® (Omalizumab), Zenapax® (Daclizumab), Zevalin® (Ibritumomab Tiuxetan), or Mylotarg® (gemtuzumab ozogamicin), Vectibix®t (panitumumab), receptors or soluble receptors, enzymes, variants, derivatives, or analogs of any of these antibodies.

The term “removing virus” or “virus removal” refers to depletion of the virus from the therapeutic protein solution, such that a fraction of the active virus particles is effectively extracted from the therapeutic protein solution. The term “inactivating” or “virus inactivation” refers to treatment of the virus containing solution with a regimen such that the contaminating viral particles are no longer infectious to cells or cannot replicate. Methods of removing and inactivating virus are discussed below.

The term “content of virus in the therapeutic protein solution is reduced” refers to a comparison of the level of virus in the therapeutic protein solution before and after the step of removing viral contaminant, as measured by DNA content, viral particle content, viral infectivity, quantitative-PCR or other means well-known in the art.

The term “isoelectric point of the virus” refers to the pH of the solution containing the virus such that the net charge of the viral protein particles has effectively been nullified in solution. Isoelectric point is determined using standard procedures in the art, including, but not limited to two-dimensional gel electrophoresis, isoelectric focusing and capillary isoelectric focusing. “About equivalent” to the isoelectric point means that the pH of the solution is near enough to the isoelectric point of the virus to allow the charge of the virus to be negligible.

Antibodies

The term “antibody” is used in the broadest sense and includes fully assembled antibodies, monoclonal antibodies, polyclonal antibodies, multispecific antibodies (e.g., bispecific antibodies), antibody fragments that can bind antigen (e.g., Fab′, F′(ab)2, Fv, single chain antibodies, diabodies), and recombinant peptides comprising the forgoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, are contemplated. Different isotypes have different effector functions; for example, IgG1 and IgG3 isotypes have antibody-dependent cellular cytotoxicity (ADCC) activity. An “immunoglobulin” or “native antibody” is a tetrameric glycoprotein composed of two identical pairs of polypeptide chains (two “light” and two “heavy” chains). The amino-terminal portion of each chain includes a “variable” (“V”) region of about 100 to 110 or more amino acids primarily responsible for antigen recognition. Within this variable region, the “hypervariable” region or “complementarity determining region” (CDR) consists of residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed. Public Health Service, National Institutes of Health, Bethesda, Md. (1991)] and/or those residues from a hypervariable loop (i.e., residues 26-32 (L1), 50-52 (L2) and 91-96 (L3) in the light chain variable domain and 26-32 (H1), 53-55 (H2) and 96-101 (H3) in the heavy chain variable domain as described by [Chothia et al., J. Mol. Biol. 196: 901-917 (1987)]. The carboxy-terminal portion of each chain defines a constant region primarily responsible for effector function.

The term “monoclonal antibody” as used herein refers to an antibody obtained from a population of substantially homogeneous antibodies, i.e., the individual antibodies comprising the population are identical except for possible naturally occurring mutations or alternative post-translational modifications that may be present in minor amounts, whether produced from hybridomas or recombinant DNA techniques. Nonlimiting examples of monoclonal antibodies include murine, chimeric, humanized, or human antibodies, or variants or derivatives thereof. Humanizing or modifying antibody sequence to be more human-like is described in, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference. One method for isolating human monoclonal antibodies is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference. Another method for isolating human monoclonal antibodies uses transgenic animals that have no endogenous immunoglobulin production and are engineered to contain human immunoglobulin loci. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); WO 91/10741, WO 96/34096, WO 98/24893, or U.S. patent application publication nos. 20030194404, 20030031667 or 20020199213; each incorporated herein by reference.

Antibody fragments may be produced by recombinant DNA techniques or by enzymatic or chemical cleavage of intact antibodies. “Antibody fragments” comprise a portion of an intact full length antibody, preferably the antigen binding or variable region of the intact antibody, and include multispecific (bispecific, trispecific, etc.) antibodies formed from antibody fragments. Nonlimiting examples of antibody fragments include Fab, Fab′, F(ab′)2, Fv [variable region], domain antibody (dAb) [Ward et al., Nature 341:544-546, 1989], complementarity determining region (CDR) fragments, single-chain antibodies (scfv) [Bird et al., Science 242:423-426, 1988, and Huston et al., Proc. Natl. Acad. Sci. USA 85:5879-5883, 1988, optionally including a polypeptide linker; and optionally multispecific, Gruber et al., J. Immunol. 152: 5368 (1994)], single chain antibody fragments, diabodies [EP 404,097; WO 93/11161; and Hollinger et al., Proc. Natl. Acad. Sci. USA, 90:6444-6448 (1993)], triabodies, tetrabodies, minibodies [Olafsen, et al., Protein Eng Des Sel. 2004 April; 17(4):315-23], linear antibodies [Zapata et al., Protein Eng., 8(10):1057-1062 (1995)]; chelating recombinant antibodies [Neri et al., J. Mol Biol. 246:367-73, 1995], tribodies or bibodies [Schoonjans et al., J. Immunol. 165:7050-57, 2000; Willems et al., J Chromatogr B Analyt Technol Biomed Life Sci. 786:161-76, 2003], intrabodies [Biocca, et al., EMBO J. 9:101-108, 1990; Colby et al., Proc Natl Acad Sci USA. 101:17616-21, 2004], nanobodies [Cortez-Retamozo et al., Cancer Research 64:2853-57, 2004], small modular immunopharmaceuticals (SMIPs) [WO03/041600, U.S. Patent publication 20030133939 and U.S. Patent Publication 20030118592], an antigen-binding-domain immunoglobulin fusion protein, a camelized antibody [Desmyter et al., J. Biol. Chem. 276:26285-90, 2001; Ewert et al., Biochemistry 41:3628-36, 2002; U.S. Patent Publication Nos. 20050136049 and 20050037421], a VHH containing antibody, or variants or derivatives thereof, and polypeptides that contain at least a portion of an immunoglobulin that is sufficient to confer specific antigen binding to the polypeptide, such as a CDR sequence, as long as the antibody retains the desired biological activity.

Non-Enveloped Virus

A non-enveloped virus refers to a virus capsid which lacks a lipid-bilayer membrane. In a non-enveloped virus, the capsid mediates attachment to and penetration into host cells. Capsids are generally either helical or icosahedral. Non-enveloped viruses range in size from 70-90 nm (Adenoviridae) to 18-26 nm (Parvoviridae). Typically small, non-enveloped viruses are extremely difficult to remove from solution. Non-enveloped viruses which can infect mammalian cells include those set out in Table 1, such as Parvoviridae, Adenoviridae, Birnaviridae, Papovaviridae (e.g., Papillomaviridae and Polyomaviridae), Picornaviridae, Reoviridae and Calciviridae.

TABLE 1

| | Enveloped/ | | | | | pH |
|---|---|---|---|---|---|---|
| Virus Family | Un-enveloped | Geno type | ds/ss^{α} | Size (nm) | pI | stability |
| Arenaviridae | E | RNA | ss | 50-300 | | s^{β} |
| Adenoviridae | U | DNA | ds | 70-90 | 5.8, 5.5-6.0 | |
| Birnaviridae | U | RNA | ds | 60-71 | | 3-9 |
| Bunyviridae | E | RNA | ss | 90-120 | | s |
| Caliciviridae | U | RNA | ss | 32-40 | 6.0-6.9 | 5-10 |
| Coronaviridae | E | RNA | ss | 60-200 | | |
| Filoviridae | E | RNA | ss | 80-14000 | | |
| Flaviridae | E | RNA | ss | 30-45 | | |
| Hepadnaviridae | E | DNA | ds | 22-42 | | |
| Herpesviridae | E | DNA | ds | 120-200 | 7.4-7.8 | |
| Iridoviridae | E | DNA | ds | 175-215 | | 4-13 |
| Orthomyxoviridae | E | RNA | ss | 80-120 | 5.0-5.3 | |
| Papillomaviridae | U | DNA | ds | 45-55 | 5.0 | |
| Paramyxoviridae | E | RNA | ss | 80-500 | | s |
| Parvoviridae | U | DNA | ss | 18-26 | 5.0-5.3 | 3-9 |
| Picornaviridae | U | RNA | ss | 20-30 | 6.1-6.4 | 3-9 |
| Poliomyelitis | | RNA | ds | | 4.5-7.5 | |
| Polyomaviridae | U | DNA | ds | 45-55 | | |
| Poxviridae | E | DNA | ds | 220-270 | 3.8-5.1 | |
| Reoviridae | U | RNA | ds | 50-70 | 3.9 | |
| Retroviridae | E | RNA | ss | 80-120 | 6.0-6.7 | |
| Rhabdoviridae | E | RNA | ss | 60-380 | | 5-10 |
| Togaviridae | E | RNA | ss | 35-70 | | |
| Toroviridae | E | RNA | ss | 120-140 | | |

^{α}ds: double stranded and ss: single stranded
^{β}s: Sensitive to low and high pH.

Other steps or procedures that may be used to remove contaminating parvovirus include a combination of flocculation of viral particles and ultrafiltration (nanofiltration) through cationic resins (Wickramasinghe et al., Biotechnol Bioeng. 86:612-21, 2004). Non-enveloped virus such as human or porcine parvovirus or human encephalomyocarditis virus (EMC) have been removed from protein solutions by addition of glycine or other amino acids, which cause aggregation of the virus particles, and subsequent nanofiltration (Yokoyama et al., Vox Sang. 86:225-9 (2004)).

Virus Inactivation and Removal

Inactivation of contaminating virus and removal of this virus is a important concern in the medical industry as production of recombinant protein and purification of proteins from plasma or other living cell components becomes the norm in the industry. The World Health Organization has recently issued guidelines and reviewed the optimal methods of inactivating and removing viruses from blood products (WHO Technical Report, Annex 4 Guidelines on viral inactivation and removal procedures intended to assure the viral safety of human blood plasma products,” Series No. 924, p 151-224, 2004). These methods are also commonly used in the purification of recombinant therapeutic proteins.

Other commonly used methods of inactivating viruses include pasteurization, detergent, heating, pH inactivation, and chemical treatment. These methods are generally successful at inactivating enveloped viruses (Wickramasinghe et al., Biotechnol Bioeng. 86:612-21 (2004)) but non-enveloped virus are more resistant to these treatments.

For example, organic solvent/detergent mixtures disrupt the lipid membrane of enveloped viruses. Once disrupted, the virus can no longer bind to and infect cells. However, non-enveloped viruses are not inactivated. Additionally, most proteins are damaged by exposure to the acidic conditions needed to kill viruses. For example, few viruses are killed at pH 5.0-5.5, a condition known to inactivate factor VIII. Immune globulin solutions are an exception. Various studies have shown that low pH, such as in the pH 4-treatment used in preparation of antibody solutions inactivates enveloped viruses (WHO Technological Report, supra). Many non-enveloped virus are resistant to this low pH treatment. Other methods of virus inactivation are available. Addition of Methylene blue to a protein solution and incubation under visible light have also been known to inactivate enveloped viruses, and may be useful to inactivate non-enveloped virus such as parvovirus (WHO Technical Report, supra; Knuever-Hopf et al., Transfusion Clin Biol, 2001, 8(Suppl 1):141 (2001)). Gamma irradiation and UVC irradiation, typically at a wavelength of 254 nm (UVC), targets nucleic acid, thus a wide variety of viruses are inactivated irrespective of the nature of their envelope (Hart et al., Vox Sang, 64:82-88 (1993); Miekka et al. Haemophilia, 1998, 4:402-408 (1998)).

Commonly used methods of virus removal include precipitation, chromatography and nanofiltration.

Precipitation with ethanol is the most widely used plasma fractionation method worldwide, although other reagents have been used. However, the contribution of ethanol to viral safety through inactivation is, marginal. Nonetheless, ethanol can also partially separate virus from protein. Viruses, as large structures, tend to precipitate at the beginning of the fractionation process when the ethanol concentration is still relatively low.

Several chromatography modes have proven very useful to remove trace amounts of impurities (e.g., DNA and endotoxin) and viruses. Among these, anion-exchange chromatography (AEX), is perhaps the most powerful. In most cases, AEX chromatography is carried out using flow-through (FT) fashion, in which impurities bind to the resin and the product of interest flows through (Li et al., Bioprocessing Journal, September/October 2005). However, the use of conventional packed-bed chromatography with FT-AEX requires columns with a very large diameter to permit high volumetric flow rates which are required to avoid a process bottleneck at the polishing step (Li et al., supra) This leads to a large column volume, which is needed for fast flow but is not optimized for binding capacity. This disadvantage with AEX columns has led to the development of membrane chromatography or membrane absorbers. Current membrane chromatography offers a convenient alternative to resin chromatography in the purification of antibodies.

Q column [e.g., Q SEPHAROSE™ (Amersham Biosciences) anion exchange resins] and Q membrane chromatography in flow through (FT) mode has proven to be a powerful viral clearance step (Zhou, et al., Biotechnology Progress 22, 341-349 (2006)). Membrane chromatography uses a micro porous membrane with ion exchange groups in the membrane pores to capture target molecules by absorption. Q membrane systems (Pall Corp., East Hills, N.Y.) employ quaternary amine functional groups in a cross-linked polymeric coating which bind negatively-charged biomolecules, such as virus particles and DNA. Q membrane chromatography and depth filtration have been developed recently for viral removal (Li et al., supra; Tipton et al., BioPharm Sept. pp. 43-50, 2002) and are innovative approaches to virus removal.

Depth filtration refers to a method of removing particles from solution using a series of filter membranes in sequence which having decreasing pore sizes. The filter membranes having the largest pore size encounter solution and particulate first and the pore size decreases as each new filter sheet is layered, establishing a gradient pore structure. The depth filter's three dimensional matrix creates a maze-like, tortuous path. The principle retention mechanisms of depth filters rely on random adsorption and mechanical entrapment throughout the depth of the matrix. The filter membranes or sheets may be wound cotton, polypropylene, rayon cellulose, fiberglass, sintered metal, porcelain or diatomaceous earth. Diatomaceous earth is a naturally-occurring soft powdery substance derived from a porous rock having microscopically-small, hollow particles. Compositions that comprise the depth filter membranes may be chemically treated to confer an electropositive charge, i.e., a cationic charge, to enable the filter to capture negatively charged particle, such as DNA, or protein aggregates. Exemplary depth filers include, but are not limited to, the A1HC filter (Millipore, Billerica, Mass.).

In anion exchange chromatography (immobilized groups are positive and bind negative ions) and cation exchange chromatography (immobilized groups are negative and bind positive ions), the pH of the protein being purified must be considered. For example, at a pH below the pI, proteins carry a net positive charge and would bind a cation exchange resin, while at a pH above the pI they carry a net negative charge and will bind to anion exchangers. The pH of an ion exchange column is determined by the pH and salt content of the buffer used for that process. Theoretically, if the ion exchange column is run with a buffer pH that is equal to the pI the protein will not exhibit strong binding to the column. In the case of virus purification or contaminant removal, Tipton et al (BioPharm Sept. p 43-50 (2002)) taught that removal of contaminating parvovirus and retrovirus by depth filtration was efficient at pH 7, which is above the pI of parvovirus thereby giving it a negative charge.

Size based nanofilter technology is perhaps the most robust viral removal unit operation currently used in pharmaceutical manufacturing. Effective removal requires that the pore size of the filter be smaller than the effective diameter of the virus. Filters with a pore size that exceeds the virus diameter may still remove some virus if it is aggregated such as by inclusion in antibody/antigen or lipid complexes. Although nanofiltration is a gentle method, proteins are subjected to shear forces that may damage their integrity and functionality. Nanometer filters can be divided into two classes: 50 and 20-nanometer pore sizes. Large pore sized filters are efficient in retaining large particle size viruses like x-MuLV and pseudorabies virus (PRV). On the other hand, filters with small pore size (20-nanometer) remove large viruses mentioned above and small virus particles such as MMV and Reo-3. In fact, in order to make membrane that can efficiently remove parvovirus such as MMV particles (18-26 nm) while at the same time providing high protein transmission, different techniques have been used by manufacturers to determine the membrane pore size. It seems the best pore size distribution for different filter membranes found is in the range of from 15 to 21 nm.

U.S. Pat. No. 6,867,285 describes a method of filtering virus from plasma-derived fibrinogen preparations comprising precipitating the protein to be purified and separating the protein from any virus using a porous membrane filter. Porous membrane filters include commercially available membranes include PLANOVA series (Asahi Kasei Corp.) having a multilayer structure comprising more than 100 layers of peripheral walls to be the membrane, VIRESOLVE series (Millipore Corp.) known as a virus removal membrane, OMEGA VR series (Pall Corporation), ULTIPOR series (Pall Corp.).

Determination of Viral Content

Viral removal or inactivation measure the clearance capacity of the purification process by determining the log reduction value (LRV) of virus, comparing the viral contaminant levels before and after the purification step, or unit operation. Determination of virus titer through viral infectivity assays is the major viral clearance evaluation method for each unit operation. All virus infectivity assays used in the process evaluation study need are validated in accordance with ICH guidelines and include proper controls for possible cytotoxic and inhibitory effects of process intermediates on the assay. The sum of the individual log₁₀ reduction factors from each unit operation represents the total viral clearance capability of the purification process.

Purification of Proteins

Purification of therapeutic proteins relies on a series of steps after harvest of cell culture media to adequately render a therapeutic protein solution pharmaceutically pure (Current Protocols in Protein Science, “Conventional chromatographic Separations,” Ch. 8-9, John Wiley & Sons Inc., Hoboken, N.J.). Generally, the steps of protein purification include capture of the protein to a more concentrated form, intermediate purification steps to remove impurities, polishing to remove additional impurities and protein variants, and virus removal, which may be done at various points during the purification process.

After initial harvest of the therapeutic protein solution from a cell culture media, usually by centrifugation of cellular debris, a capture step is performed. Common methods of capture include affinity chromatography and size exclusion chromatography. Affinity chromatography relies on the affinity of the protein being purified for a another molecule bound to the resin in the column, such as a ligand for a receptor or an antibody or agents that bind certain types of proteins, such as bacterially-derived Protein A and Protein G molecules. Gel filtration or size exclusion chromatography separates proteins on the basis of size of the protein. Additional capture processes are known in the art and may be applied to capture the protein of interest.

Intermediate purification steps are useful to remove other biomolecules such as protein or DNA/RNA contaminants, small cellular debris, and the like (Current Protocols in Protein Science, “Conventional Chromatographic Separations,” Ch. 8, John Wiley & Sons Inc., Hoboken, N.J.).

Polishing steps are used to remove impurities such as structural and functional variants of the protein of interest, from protein solutions that are not eliminated during the capture process. These impurities include protein aggregates, host cell protein debris, nucleic acids, leached capture agent, such as Protein A or Protein G, and potential viral contaminants. Processes useful as polishing steps include cation-exchange chromatography, anion-exchange chromatography, hydrophobic-interaction chromatography, and ceramic hydroxyapatite chromatography (Li et al., BioProcessing Journal September/October 2005, pp 1-8), as well as reverse-phase HPLC, gel filtration, affinity chromatography or chromatofocusing (Current Protocols in Protein Science, John Wiley & Sons Inc.). Affinity chromatography includes, but is not limited to, purification using lectin affinity, dye affinity, ligand affinity, metal-chelate affinity, immunoaffinity, affinity tags and sequence-specific DNA binding affinity.

Cation-exchange chromatography (CEX) is a useful tool remove host cell protein and DNA, aggregate proteins, excess capture agent, and some viruses. CEX resin provides high product binding capacity at a high conductivity and high resolution to remove tarter protein variants.

Anion exchange chromatography (AEX) is useful as a polishing step to remove host cell protein and DNA, aggregate proteins, excess capture agent, and some viruses. AEX is typically carried out using flow-through methods, in which impurities bind to the resin and the product of interest flows through the column. This can lead to problems obtaining adequate columns, leading to the development of AEX membrane chromatography, e.g., Q membrane technology.

In hydrophobic-interaction chromatography (HIC), proteins are separated based on the strength of the proteins hydrophobic interaction to hydrophobic groups (e.g. phenyl-, octyl groups) attached to column resin. The variation in hydrophobicity from one protein species to another makes it possible to selectively adsorb proteins on an HIC column (Current Protocols in Protein Science, “Conventional chromatographic Separations,” Ch. 8.4, 1995, John Wiley & Sons Inc., Hoboken, N.J.). Hydroxyapatite is a form of calcium phosphate useful to purify proteins and nucleic acids. Protein binding to hydroxyapatite is mediated by interactions between the amino and carboxy groups on the protein and the calcium and phosphate groups on the matrix (Current Protocols in Protein Science, “Conventional chromatographic Separations,” Ch. 8.5, 1997, John Wiley & Sons Inc., New Jersey). Hydrophobic-interaction chromatography and ceramic hydroxyapatite efficiently remove protein dimers and larger aggregates using either bind and elute methods or flow-through methods.

Chromatofocusing (CF) separates proteins based on the protein's isoelectic point (pI). Proteins elute from a CF column in descending order of pI due to the descending linear pH gradient used to elute the proteins from the column. (Current Protocols in Protein Science, “Conventional chromatographic Separations,” Ch. 8.6, 1995, John Wiley & Sons Inc., New Jersey). The efficacy of chromatofocusing relies on the pH range of the buffers for protein elution, which usually span up to several pH units above and below the pH of the protein of interest.

An exemplary protein purification and virus removal process are demonstrated in purification of therapeutic monoclonal antibodies. The Mab large-scale purification process is usually built around the employment of immobilized Protein A as the primary capture and purification step in combination with other column operations. The entire process consists of three or four purification units, which include harvest/recovery and two to three ‘polishing’ purification units (Li et al., supra). The chromatographic polishing steps remove product-related impurities, such as cell lysis components, and potentially provide some degree of viral clearance. The process typically also includes viral removal by filtration, low pH viral inactivation, cross flow filtration for buffer exchange and concentration, and 0.2 μm sterile filtration. A low pH elution buffer is needed in order to remove and collect purified Mabs from protein A affinity resin. The pH of the elution buffer solution commonly used ranges from pH 3.0 to 3.4, and the pH of protein A elution pool ranges from 3.6 to 4.2 depending on the buffer ionic strength.

Except in the cross flow and sterile filtrations, each unit operation is validated with/by viral clearance studies using the appropriate scale down model. Although the above methodologies are useful for removal of viral contaminants, no one methodology stands out as an optimal process. Thus, there is a need to develop additional processes for removal of viral contaminants from therapeutic protein solutions.

Additional aspects and details of the invention will be apparent from the following examples, which are intended to be illustrative rather than limiting.

### EXAMPLES

### Example 1

Murine Minute Virus, a non-enveloped single-strand DNA parvovirus with an average size of 18-26 nm, is a difficult viral species to be killed or inactivated. Due to its properties, survival ability and particle size, MMV is used as one of model viruses for the validation of a provide bioprocess. To determine a more efficient method of removing this viral contaminant from protein purification processes, a method of removing virus using depth filtration was developed.

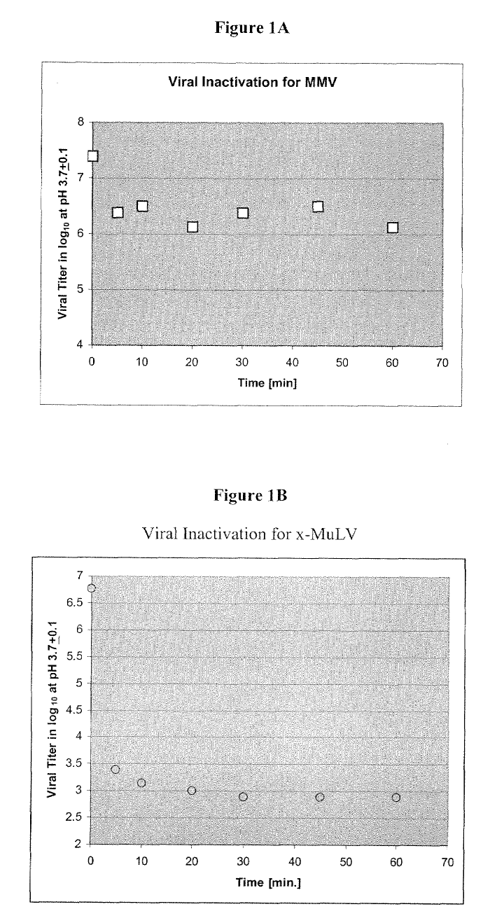

Initially, culture media containing a monoclonal antibody (Mab) was passed over a protein A column to purify the protein from the culture media using a standard procedures known in the art (Schule et al., J. Chromatogr. 587:61-70, (1991)). The Mab was then eluted from the Protein A column using elution buffer according to the manufacturers instructions [e.g., GE Healthcare, Millipore PROsept VAO, Applied Biosystems, PoroA], using a low pH buffer (for example, pH 3.4, 50-100 mm acetic acid). The collected eluate from the Protein A column pool, typically having a pH about 4.2, was warmed to room temperature and titrated with 3M Tris base (pH 10.5) to pH 3.7±0.1. The volume of Tris used for titration is about 2% of the total Protein A pool volume. The titrated pool is maintained at room temperature for 60 to 75 minutes and viral clearance measured. Viral clearance data indicated that this step is not efficient to kill naked viruses such as MMV particles; however, the enveloped viruses such as x-MuLV particles are inactivated in 60 minutes. The typical MMV and x-MuLV viral inactivation in the low pH treatment are illustrated in FIGS. 1A and 1B, respectively. These figures illustrate that MMV titer is reduced only approximately one log after low pH activation while x-MuLV is reduced by approximately 4 logs at low pH after 60 minutes.

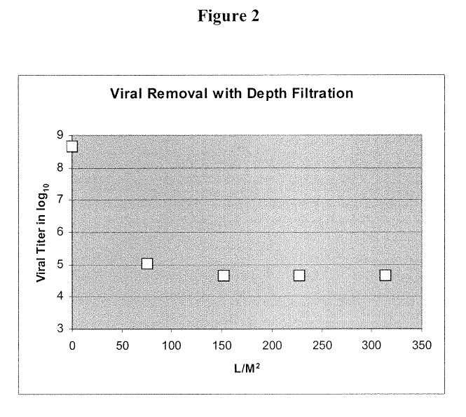

After low pH treatment, the PVINP pool (Protein Viral Inactivation Pool) is titrated to pH 5.0 in room temperature with 10% acidic acid (about 2% of total pool volume). An A1HC pod depth filter from Millipore (Billerica, Mass.) is used to clarify the pool turbidity. Results demonstrated that A1HC filter consistently removed CHOP particulate, decreasing levels from over 6000 ppm to <100 ppm, and removed DNA from over 10,000 ppb to less than 10 ppb in six reproducibility runs. In addition, A1HC at pH 5, efficiently showed approximately a 3-4 log reduction value of naked DNA MMV viruses and a 3 log reduction value of naked RNA PRV viruses. The operation was performed at a flow rate of 216 LMH and process capacity of 300 L/m2. FIG. 2 shows a typical MMV removal with A1HC depth filter.

These results demonstrated that the A1HC depth filter (Millipore) was able to efficiently remove MMV virus particles with a 4 LRV at pH 5.0 MMV [highly hydrophobic] and 1.96 LRV for MuLV [high negative charged] from the Mab pool of Protein A affinity chromatography post low pH viral inactivation (Table 2).

TABLE 2
Depth Filtration for Viral Clearance for Antibody solution

| Purification Unit | Resin or others | Condition | MMV_LRV | MuLV_LRV | Protein |
|---|---|---|---|---|---|
| Depth Filtration | A1HC | pH 5.0 | 4 | 1.9 | MAb |
| Post Viral Inactivation | | | | | |

The accumulated process data continuously demonstrated the robustness and consistency for DNA and CHOP (Chinese hamster ovary protein) removal by using A1HC depth filtration (Table 3A and 3B). Thus, the data reflects the robustness and consistency of MMV removal by depth filtration.

TABLE 3A
Summary-Capture Process with Protein A

| Steps | Yield % | CHOP ppm | DNA ppm |
|---|---|---|---|
| | Lot 1 | | |
| Mab Pool | 105.2 | 1.31E+03 | 4.03E+03 |
| VI Pool | | | |
| Filtered VI Pool | 91.7 | 13.73 | 0.86 |
| | Lot 2 | | |
| Mab Pool | 99 | 1.50E+03 | 2.60E+03 |
| VI Pool | 97.3 | 1.26E+03 | 2.27E+03 |
| Filtered VI Pool | 94.6 | 26.3 | 0.99 |
| I | Lot 3 | | |
| Mab Pool | 100.1 | 1.44E+03 | 3.43E+03 |
| VI Pool | 97.4 | 1.15E+03 | 2.75E+03 |
| Filtered VI Pool | 96.4 | 18.4 | 0.79 |

TABLE 3B
Summary-Capture Process with Protein A

| | Lot 4 | | | Lot 5 | | |
|---|---|---|---|---|---|---|
| | | CHOP | | Yield | CHOP | DNA |
| Steps | Yield % | ppm | DNA ppm | % | ppm | ppm |
| Mab Pool | 98 | 2.00E+03 | 1.10E+04 | 101 | 9.00E+02 | ND |
| VI Pool | 100 | | | 100 | | |
| Filtered VI | 100 | 31 | 0.66 | 100 | 8.6 | ND |
| Pool | | | | | | |

A low pH viral inactivation at 3.7±1 in our experiments indicated that the step is not efficient for inactivation of MMV [1.26 LRV] but efficient for inactivation of MuLV [3.8 LRV] (FIGS. 1, A and B). However, the low pH viral inactivation is achieved by chemical-solution titration and physical settings for incubation time and temperature, the system is considered as consistent and robust.

Additional experiments confirm that the low pH step combined with the depth filtration are efficient for small non-enveloped viruses. For example, large viruses pseudorabies virus (PRV) and MuLV are removed efficiently during the low pH inactivation step, whereas small viruses MMV and Reo virus (˜50 nm) are not removed by low pH inactivation (Table 4).

TABLE 4

| | Virus | | | |
|---|---|---|---|---|
| Step | PRV | x-MuLV | MMV | Reo 3 |
| Low pH | 3.33 | 2.59 | 0.13 | 0.22 |
| A1HC | 3.69 | 2.57 | 2.91 | 3.97 |

The low viral inactivation efficiency at this step for MMV combined with the depth filtration at pH 5.0 based the results provides a consistent MMV clearance about a total of 6 LRV and a MuLV clearance about 6 LRV, respectively. Therefore, the combination of the low pH viral inactivation and pH A1HC depth filtration for potential MMV and MuLV clearance power is enhanced.

Although the use of a depth filter is currently not recognized by the regulatory agencies as a robust orthogonal method for virus removal, the utilization of the A1HC membrane in the purification process provides additional safety confidence to purification processes.

Numerous modifications and variations in the invention as set forth in the above illustrative examples are expected to occur to those skilled in the art. Consequently only such limitations as appear in the appended claims should be placed on the invention

What is claimed:

1. A method for removing parvovirus or fragments thereof from a therapeutic protein solution comprising the step of:
passing the solution through a depth filter at a pH within 1 pH unit of the isoelectric point (pI) of said virus.

2. The method of claim 1 wherein the pH is within the range of pH 4.0 to pH 6.

3. The method of claim 1 wherein the pH is about pH 5.

4. The method of claim 1 wherein the virus is selected from the group consisting of mouse minute virus, mouse parvovirus, porcine parvovirus and human parvovirus.

5. The method of claim 1 wherein the average size of the virus is less than about 30 nm.

6. The method of any of claims 1-5 further comprising the step of maintaining the solution at a pH and for a length of time effective to inactivate virus in the solution.

7. The method of any of claims 1-6, wherein the content of parvovirus in the therapeutic protein solution is reduced by at least 2 logs.

8. The method of claim 7 wherein the parvovirus content of the therapeutic protein solution is reduced by 5 logs.

9. The method of any of claims 1-8 wherein the depth filter comprises diatomaceous materials.

10. The method of any of claims 1-9 wherein the depth filter is an electropositively charged filter.

11. The method of claim 9 wherein the depth filter is a Millipore A1HC filter.

12. The method of claim 6 wherein the pH inactivating step is carried out at a pH within the range of pH 2.5 to pH 5.

13. The method of claim 6 wherein the inactivating step is from 15 to 90 minutes.

14. The method of any of claims 1-13 wherein the protein is an antibody.

15. The method of claim 14 wherein the solution is passed through a protein A affinity chromatography column before being passed through the depth filter.

16. The method of claim 15 wherein the protein A affinity chromatography step is carried out before the pH inactivation step, and wherein the pH activation step is carried out before the depth filtration step.
